(19) United States
(12) Patent Application Publication
(10) Pub. No.: US 2009/0005813 A1
(43) Pub. Date: Jan. 1, 2009

(54) APPARATUS AND METHODS FOR SPINAL IMPLANTS

(76) Inventors: ANGELA CRALL, Austin, TX (US); Charles Forton, Leander, TX (US); Rodney Young, Austin, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/770,329

(22) Filed: Jun. 28, 2007

(51) Int. Cl. A61B 17/70; A61B 17/56; A61B 17/04; A61B 17/58

(52) U.S. Cl. 606/246; 606/250; 606/264; 606/301; 606/305

(57) ABSTRACT

A polyaxial screw device and system for spinal fixation and dynamic stabilization. Embodiments of the present disclosure may utilize hinged connections, cam-style mechanisms, and planar connectors to enable surgeons to attach to an implanted bone fastener or connect to an elongated member from a lateral approach for improved accessibility, with a low-profile configuration for improved patient comfort. Embodiments may be implantable using MIS procedures and techniques.

### TECHNICAL FIELD OF THE DISCLOSURE

This disclosure relates generally to spinal implants, and more particularly to bone anchoring elements for attaching an elongated member to a vertebra.

### BACKGROUND OF THE DISCLOSURE

Modern spine surgery often involves spinal fixation through the use of spinal implants or fixation systems to correct or treat various spine disorders or to support the spine. Spinal implants may help, for example, to stabilize the spine, correct deformities of the spine, facilitate fusion, or treat spinal fractures. A spinal fixation system typically includes corrective spinal instrumentation that is attached to selected vertebra of the spine by screws, hooks, and clamps. The corrective spinal instrumentation includes spinal rods or plates that are generally parallel to the patient's back. The corrective spinal instrumentation may also include transverse connecting rods that extend between neighboring spinal rods. Spinal fixation systems are used to correct problems in the cervical, thoracic, and lumbar portions of the spine, and are often installed posterior to the spine on opposite sides of the spinous process and adjacent to the transverse process.

Often, spinal fixation may include rigid (i.e., in a fusion procedure) support for the affected regions of the spine. Such systems limit movement in the affected regions in virtually all directions (for example, in a fused region). More recently, so called “dynamic” systems have been introduced wherein the implants allow at least some movement of the affected regions in at least some directions, i.e. flexion, extension, lateral bending, or torsion. In many of these systems, dynamic movement is enabled through the use of flexible rods.

### SUMMARY OF THE DISCLOSURE

Embodiments of the present disclosure offer configurations for connecting elongated members and other spinal fixation members to pedicle screws, bone fasteners and the like, without the need for a threaded cap or threaded insert to retain the construct, thereby providing surgeons with devices for alternative solutions. Furthermore, embodiments of the present disclosure do not have the downward pressure associated with threading a cap or other device onto the construct, so there is less stress on the patient.

Embodiments of the present disclosure also enable the surgeon to approach the fixation site from a lateral approach instead of the traditional superior approach, further providing surgeons different solutions to methods for implanting the devices.

Embodiments of the present disclosure further may be implanted after the bone fastener and an elongated member are in place, thereby enabling surgeons to augment previously implanted systems.

Components of spinal stabilization systems may be made of materials including, but not limited to, titanium, titanium alloys, stainless steel, ceramics, and/or polymers. Some components of a spinal stabilization system may be autoclaved and/or chemically sterilized. Components that may not be autoclaved and/or chemically sterilized may be made of sterile materials. Components made of sterile materials may be placed in working relation to other sterile components during assembly of a spinal stabilization system.

In accordance with one feature of the disclosure a collar is provided for use with polyaxial screws in an implant system that supports a spine. The collar may be useful for either fusion or dynamic stabilization situations. As one feature, the collar may connect to the bone fastener after the bone fastener has been implanted in the vertebra and optionally after the elongated member has been positioned proximate the bone fastener. As one feature, the collar may be configured to capture a bone fastener and a spinal fixation member from a lateral approach, enabling spinal fixation procedures directed at angles other than at the vertebra.

One embodiment of the present disclosure is generally directed to a collar for connecting an elongated member to pre-positioned bone fasteners having two members. Each member may be configured for hingedly connecting to the other member. Recessed portions in each member may align with corresponding recessed portions to form a cavity when the members are connected. The cavity may extend all the way through the collar to receive an elongated member, or may extend a certain depth and have a selected profile to receive a portion of a bone fastener. The collar may also have a connector to secure the first and second connectors together once the elongated member and bone fastener have been captured. Two or more recessed portions may be communicably joined to define a channel and two legs. The two legs may have interior surfaces adapted for capturing a portion of the bone fastener and a portion of the elongated member. The upper portion of the collar may have a substantially curved surface.

Another embodiment of the collar may have a curved outer surface having a major diameter and a minor diameter. The collar may include a cavity having selected depth and profile for receiving a portion of a bone fastener and an elongated member channel defining a depth and profile for receiving a portion of an elongated member. The sides of the channel may form two upwardly extending legs. The legs may further be configured with outwardly extending flanges or inwardly extending notches. The collar may also include a cap having a curved inner surface with a major diameter greater than the major diameter of the curved outer surface of the collar and a minor diameter less than the major diameter of the curved outer surface of the collar, and may further include flanges to engage flanges or notches on the collar. The action of rotating the cap onto the collar may compress the legs inward to retain the elongated member in a selected configuration with the bone fastener. Furthermore, embodiments may have a plurality of radially extending features on the top surface of the collar for contact with a plurality of radially extending features on the bottom surface of the cap to maintain the cap in a selected orientation with the collar. The cap and/or the upper portion of the collar may also have a curved upper surface for reduced profile.

Yet another embodiment is directed to a collar for connection between an elongated member and a bone fastener implanted in bony tissue. A collar may include a cavity for attachment to a portion of a bone fastener implanted in bony tissue. A collar may include a channel for receiving a portion of an elongated member. The channel may form two upwardly extending legs. Each leg may have a rectilinear opening in alignment with a rectilinear opening on the opposite leg. A planar connector may be configured for insertion into the two openings such that the elongated member is maintained in selected configuration with the bone fastener. The planar connector may have a curved profile configured for increased contact with the elongated member. The planar connector may be compression fit into the two openings, or sweat-locked into the two openings.

These, and other, aspects of the disclosure will be better appreciated and understood when considered in conjunction with the following description and the accompanying drawings. The following description, while indicating various embodiments of the disclosure and numerous specific details thereof, is given by way of illustration and not of limitation. Many substitutions, modifications, additions or rearrangements may be made within the scope of the disclosure, and the disclosure includes all such substitutions, modifications, additions or rearrangements.

### BRIEF DESCRIPTION OF THE FIGURES

FIG. 1 depicts a perspective view of an embodiment of a bone stabilization system;

FIG. 2 depicts a perspective view of one embodiment of a bone fastener;

FIG. 3 depicts a cross-sectional view of one embodiment of a polyaxial bone fastener assembly according to one embodiment of the present disclosure;

FIG. 4 depicts a cross-sectional view of one embodiment of a polyaxial screw collar according to one embodiment of the present disclosure;

FIG. 5 depicts a cross-sectional view of one embodiment of a polyaxial screw collar according to one embodiment of the present disclosure;

FIG. 6A depicts an exploded view of a cam locking collar according to one embodiment of the present disclosure;

FIG. 6B depicts a bottom view of a closure member for a cam-locking collar according to one embodiment of the present disclosure;

FIG. 6C depicts a side view of a cam locking collar according to one embodiment of the present disclosure;

FIG. 7A depicts a side view of a collar according to one embodiment of the present disclosure;

FIG. 7B depicts a perspective view of a collar according to one embodiment of the present disclosure; and

FIG. 7C depicts a perspective view of a planar connector according to one embodiment of the present disclosure;

FIG. 7D depicts a perspective view of an alternate planar connector according to one embodiment of the present disclosure.

### DETAILED DESCRIPTION OF THE PREFERRED EMBODIMENTS

The disclosure and the various features and advantageous details thereof are explained more fully with reference to the non-limiting embodiments that are illustrated in the accompanying drawings and detailed in the following description. Descriptions of well known starting materials, processing techniques, components and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, additions or rearrangements within the scope of the underlying inventive concept(s) will become apparent to those skilled in the art after reading this disclosure.

A spinal stabilization system may be installed in a patient to stabilize a portion of a spine. Spinal stabilization may be used, but is not limited to use, in patients having degenerative disc disease, spinal stenosis, spondylolisthesis, pseudoarthrosis, and/or spinal deformities; in patients having fracture or other vertebral trauma; and in patients after tumor resection. A spinal stabilization system may be installed using a minimally invasive procedure. An instrumentation set may include instruments and spinal stabilization system components for forming a spinal stabilization system in a patient.

A minimally invasive procedure may be used to limit an amount of trauma to soft tissue surrounding vertebrae that are to be stabilized. In some embodiments, the natural flexibility of skin and soft tissue may be used to limit the length and/or depth of an incision or incisions needed during the stabilization procedure. Minimally invasive procedures may provide limited direct visibility in vivo. Forming a spinal stabilization system using a minimally invasive procedure may include using tools to position system components in the body.

A minimally invasive procedure may be performed after installation of one or more spinal implants in a patient. The spinal implant or spinal implants may be inserted using an anterior procedure and/or a lateral procedure. The patient may be turned and a minimally invasive procedure may be used to install a posterior spinal stabilization system. A minimally invasive procedure for stabilizing the spine may be performed without prior insertion of one or more spinal implants in some patients. In some patients, a minimally invasive procedure may be used to install a spinal stabilization system after one or more spinal implants are inserted using a posterior spinal approach.

A spinal stabilization system may be used to achieve rigid pedicle fixation while minimizing the amount of damage to surrounding tissue. In some embodiments, a spinal stabilization system may be used to provide stability to two adjacent vertebrae (i.e., one vertebral level). A spinal stabilization system may include two bone fastener assemblies. One bone fastener assembly may be positioned in each of the vertebrae to be stabilized. An elongated member may be coupled and secured to the bone fastener assemblies. As used herein, “coupled” components may directly contact each other or may be separated by one or more intervening members. In some embodiments, a single spinal stabilization system may be installed in a patient. Such a system may be referred to as a unilateral, single-level stabilization system or a single-level, two-point stabilization system. In some embodiments, two spinal stabilization systems may be installed in a patient on opposite sides of a spine. Such a system may be referred to as a bilateral, single-level stabilization system or a single-level, four-point stabilization system.

In some embodiments, a spinal stabilization system may provide stability to three or more vertebrae (i.e., two or more vertebral levels). In a two vertebral level spinal stabilization system, the spinal stabilization system may include three bone fastener assemblies. One bone fastener assembly may be positioned in each of the vertebrae to be stabilized. An elongated member may be coupled and secured to the three bone fastener assemblies. In some embodiments, a single two-level spinal stabilization system may be installed in a patient. Such a system may be referred to as a unilateral, two-level stabilization system or a two-level, three-point stabilization system. In some embodiments, two three-point spinal stabilization systems may be installed in a patient on opposite sides of a spine. Such a system may be referred to as a bilateral, two-level stabilization system or a two-level, six-point stabilization system.

In some embodiments, combination systems may be installed. For example, a two-point stabilization system may be installed on one side of a spine, and a three-point stabilization system may be installed on the opposite side of the spine. The composite system may be referred to a five-point stabilization system.

Minimally invasive procedures may reduce trauma to soft tissue surrounding vertebrae that are to be stabilized. Only a small opening may need to be made in a patient. For example, for a single-level stabilization procedure on one side of the spine, the surgical procedure may be performed through a 2 cm to 4 cm incision formed in the skin of the patient. In some embodiments, the incision may be above and substantially between the vertebrae to be stabilized. In some embodiments, the incision may be above and between the vertebrae to be stabilized. In some embodiments, the incision may be above and substantially halfway between the vertebrae to be stabilized. Dilators, a targeting needle, and/or a tissue wedge may be used to provide access to the vertebrae to be stabilized without the need to form an incision with a scalpel through muscle and other tissue between the vertebrae to be stabilized. A minimally invasive procedure may reduce an amount of post-operative pain felt by a patient as compared to invasive spinal stabilization procedures. A minimally invasive procedure may reduce recovery time for the patient as compared to invasive spinal procedures.

Spinal stabilization systems may be used to correct problems in lumbar, thoracic, and/or cervical portions of a spine. Various embodiments of a spinal stabilization system may be used from the C1 vertebra to the sacrum. For example, a spinal stabilization system may be implanted posterior to the spine to maintain distraction between adjacent vertebral bodies in a lumbar portion of the spine.

In some embodiments, an elongated member and bone fastener may be maintained in a fixed configuration desirable for spinal fusion, and in other embodiments, an elongated member and a bone fastener may be maintained in movable contact desirable for dynamic stabilization to maintain spinal functionality. To accomplish these goals, in some embodiments, the collar may allow limited sliding and rotation of the elongated member relative to the bone fastener to provide some range of spinal bending, preferably flexion/extension motion, with the range of spinal bending preferably being sufficient to assist the adequate supply of nutrients to a disc in the supported portion of the spine.

Reference is now made in detail to the exemplary embodiments of the disclosure, examples of which are illustrated in the accompanying drawings. Wherever possible, the same reference numbers will be used throughout the drawings to refer to the same or like parts (elements).

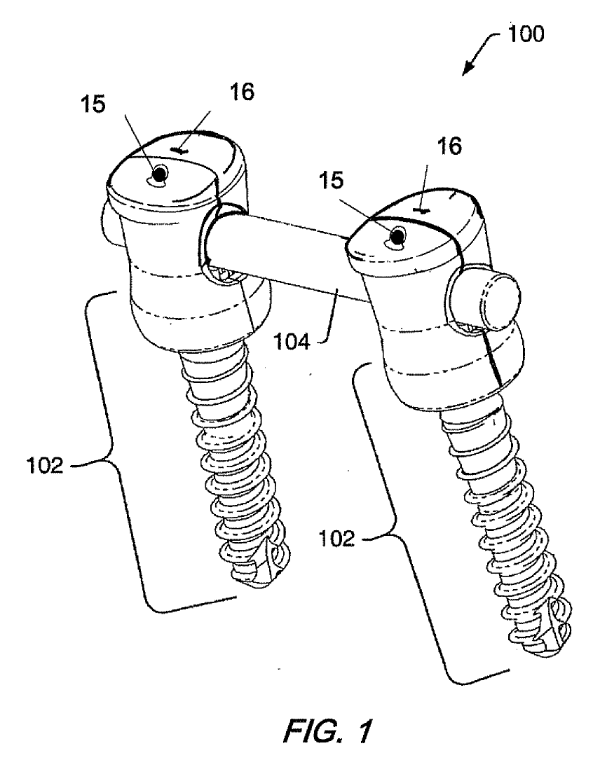

FIG. 1 depicts an embodiment of spinal stabilization system 100 that may be implanted using a minimally invasive surgical procedure. Spinal stabilization system 100 may include bone fastener assemblies 102 and elongated member 104. Other spinal stabilization system embodiments may include, but are not limited to, plates, dumbbell-shaped members, and/or transverse connectors FIG. 1 depicts a spinal stabilization system for one vertebral level. In some embodiments, the spinal stabilization system of FIG. 1 may be used as a multi-level spinal stabilization system if one or more vertebrae are located between the vertebrae in which bone fastener assemblies 102 are placed. In other embodiments, multi-level spinal stabilization systems may include additional bone fastener assemblies to couple to one or more other vertebrae.

A bone fastener may be, but is not limited to, a bone screw, a ring shank fastener, a barb, a nail, a brad, or a trocar. Bone fasteners and/or bone fastener assemblies may be provided in various lengths in an instrumentation set to accommodate variability in vertebral bodies. For example, an instrumentation set for stabilizing vertebrae in a lumbar region of the spine may include bone fastener assemblies with lengths ranging from about 30 mm to about 75 mm in 5 mm increments. A bone fastener assembly may be stamped with indicia (i.e., printing on a side of the collar). In some embodiments, a bone fastener assembly or a bone fastener may be color-coded to indicate a length of the bone fastener. In certain embodiments, a bone fastener with a 30 mm thread length may have a magenta color, a bone fastener with a 35 mm thread length may have an orange color, and a bone fastener with a 55 mm thread length may have a blue color. Other colors may be used as desired.

Each bone fastener provided in an instrumentation set may have substantially the same thread profile and thread pitch. In an embodiment, the thread may have about a 4 mm major diameter and about a 2.5 mm minor diameter with a cancellous thread profile. In certain embodiments, the minor diameter of the thread may be in a range from about 1.5 mm to about 4 mm or larger. In certain embodiments, the major diameter of the thread may be in a range from about 3.5 mm to about 6.5 mm or larger. Bone fasteners with other thread dimensions and/or thread profiles may also be used. A thread profile of the bone fasteners may allow bone purchase to be maximized when the bone fastener is positioned in vertebral bone.

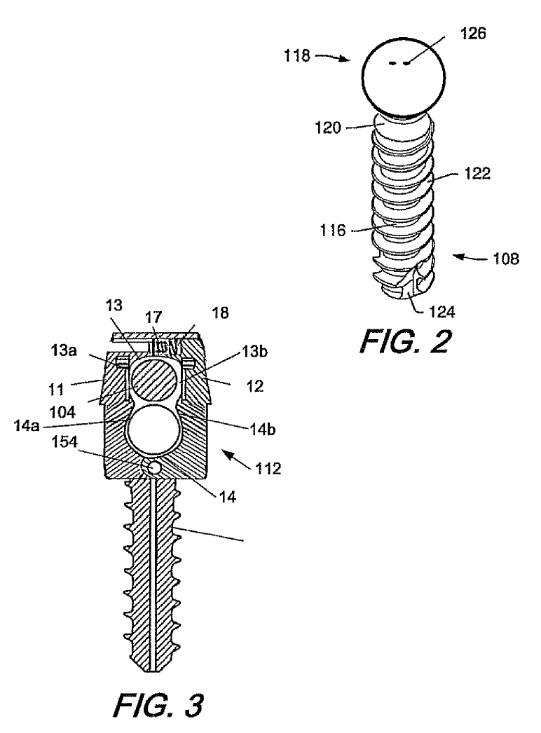

FIG. 2 depicts a perspective view of one embodiment of a bone fastener. Bone fastener 108 may include shank 116, head 118, and neck 120. Shank 116 may include threading 122. In some embodiments, threading 122 may include self-tapping start 124. Self-tapping start 124 may facilitate insertion of bone fastener 108 into vertebral bone.

Head 118 of bone fastener 108 may include various configurations to engage a driver that inserts the bone fastener into a vertebra. In some embodiments, the driver may also be used to remove an installed bone fastener from a vertebra. In some embodiments, head 118 may include one or more tool portions 126. Tool portions 126 may be recesses and/or protrusions designed to engage a portion of the driver. In some embodiments, bone fastener 108 may be cannulated for use in a minimally invasive procedure.

Head 118 of bone fastener 108 may have a spherical profile, as depicted in FIG. 2. Neck 120 of bone fastener 108 may have a smaller diameter than adjacent portions of head 118 and shank 116. The diameter of neck 120 may fix the maximum angle that the collar of the bone fastener assembly can be rotated relative to bone fastener 108. In some embodiments, neck 120 may be sized to allow up to about 40 degrees or more of angulation of the collar relative to the bone fastener. In some embodiments, the neck may be sized to allow up to about 30 degrees of angulation of the collar relative to the bone fastener. In some embodiments, the neck may be sized to allow up to about 20 degrees of angulation of the collar relative to the bone fastener. A bone fastener may be rotatably positioned in a collar such that the bone fastener is able to move radially and/or rotationally relative to the collar (or the collar relative to the bone fastener) within a defined range of motion. The range of motion may be provided within a plane, such as by a hinged connection, or within a three-dimensional region, such as by a ball and socket connection. Motion of the bone fastener relative to the collar (or the collar relative to the bone fastener) may be referred to as “angulation” and/or “polyaxial movement”.

Referring to FIG. 3, a collar 112 useful for maintaining an elongated member 104 in a selected configuration with a bone fastener 108 may include first member 11 connected with hinge 154 to second member 12. Hinged connections may be made through use of a hinge bracket on either member rotatably connected to a hinge pin on the other member.

In an embodiment, a hinge bracket and hinge pin may be manufactured separately and joined to form hinge 154 to hingedly connect first member 11 with second member 12. In other embodiments a hinge bracket and hinge pin may be integral to first member 11 or second member 12 to form hinge 154 to provide the same functionality. A hinge bracket may rotate about a longitudinal axis of a hinge pin such that the hinge pin may be capable of rotating a select angle about the hinge bracket, thereby rotatably connecting first member 11 and second member 12 to form collar 112. First member 11 and second member 12 may be rotated about hinge 154 formed by the hinge pin rotating in the hinge bracket. First member 11 and second member 12 may be rotated about hinge 154 to capture a head of a bone fastener 108 in an opening. First member 11 and second member 12 may be rotated about hinge 154 to capture a portion of an elongated member 104.

Collar 112 may include a closure member for capturing, locking, or otherwise maintaining a connection between first member 11 and second member 12 such that elongated member 104 and bone fastener 108 may be maintained in a selected configuration. In some embodiments, the closure member for connecting first member 11 to second member 12 may be threaded, such as a threaded member 17 threaded into threaded cavity 18 depicted in FIG. 3. When collar 112 is in a closed configuration as shown in FIG. 3, the threaded member 17 may enter first member 11 and advance into threaded cavity 18 in second member 12 to securely engage first member 11 to second member 12. Advantageously, the action of tightening a threaded member as depicted in FIG. 3 may be achieved without applying pressure directly to the spine, but may instead be achieved by applying tangential forces and actions.

In one embodiment of a collar 112, a first member 11 includes a first recessed portion 13a, and a second member 12 includes a first recessed portion 13b aligned such that the first recessed portion 13a on first member 11 and the first recessed portion 13b on second member 12 form first cavity 13 to capture a portion of elongated member 104.

Furthermore, the threaded member 17 may enter first member 11 and advance into a cavity in second member 12 to securely engage first member 11 to second member 12 such that the alignment of a second recessed portion 14a on first member 11 and a second recessed portion 14b on second member 12 form a second cavity 14 to capture head 118 of bone fastener 108.

First recessed portions 13a and 13b may be bored, milled, or otherwise machined from collar 112 to have the desired profile, such as a radius or width, to accommodate elongated member 104. Alternatively, first member 11 and second member 12 may be formed during a casting process, welded, or otherwise built up to include recessed portions 13a and 13b. First recessed portions 13a and 13b may further include a layer for contact with elongated member 104, biocompatibility, or other functionality. For example, a portion of recessed portions 13a and 13b may be bead blasted, knurled, or otherwise adapted for higher friction for higher spinal rigidity, or may be polished smooth or coated with UHMWPE, PEEK or other material for a reduced friction coefficient, thus increasing spinal flexibility.

Other recessed portions may be bored, milled, or otherwise machined from first and second members 11 and 12 to form a second cavity with a desired profile when first and second members 11 and 12 are in a closed configuration. For example, second recessed portions 14a and 14b may be manufactured having a selected radius or width to accommodate head 118 of bone fastener 108. Alternatively, first member 12 may be formed during a casting process, welded, or otherwise built up to include second recessed portions. A second cavity may further include a layer selected based on the contact with head 118 of bone fastener 108, biocompatibility, or other functionality. For example, a layer in a second cavity may be bead blasted, knurled, or otherwise adapted for higher friction for higher spinal rigidity, or may be coated with UHMWPE or PEEK for a reduced friction coefficient for more spinal flexibility.

The embodiment shown in FIG. 3 may provide a secure construct to maintain elongated member 104 in a selected configuration with respect to bone fastener 108 without requiring a closure member vertically threaded into collar 112 to maintain elongated member 104 in contact with bone fastener 108. One advantage of avoiding the use of threaded closure members to secure elongated member 104 in contact with bone fastener 108 is that the upper portion of the bone fastener assembly 100 (i.e. the part of collar 112 that extends above elongated member 104) may define a curved surface or otherwise less angular profile than prior art collars that require a cap or plug threaded to the collar. A second effect is that collar 112 may be manufactured with a lower profile than prior art collars, which typically require a minimum number of threads to securely engage a cap or other threaded closure member. Also, the action of engaging the closure member may be achieved by applying forces tangential or otherwise not directed at the spinal column. Those skilled in the art will appreciate that the lower profile, curved upper surfaces, and avoidance of vertically threaded closure members may provide a patient with more comfort and less pain and may have a reduced risk of damaging tissues and organs near the stabilization site.

First cavity 13 may extend through collar 112 or may extend a selected depth and profile into collar 112. In one embodiment, a second cavity 14 may have a selected depth and profile to capture the head of a bone fastener such as bone fastener 108 depicted in FIG. 2. Furthermore, cavities such as cavity 13 may have constant cross-sectional profile or may have a symmetric or asymmetric profile.

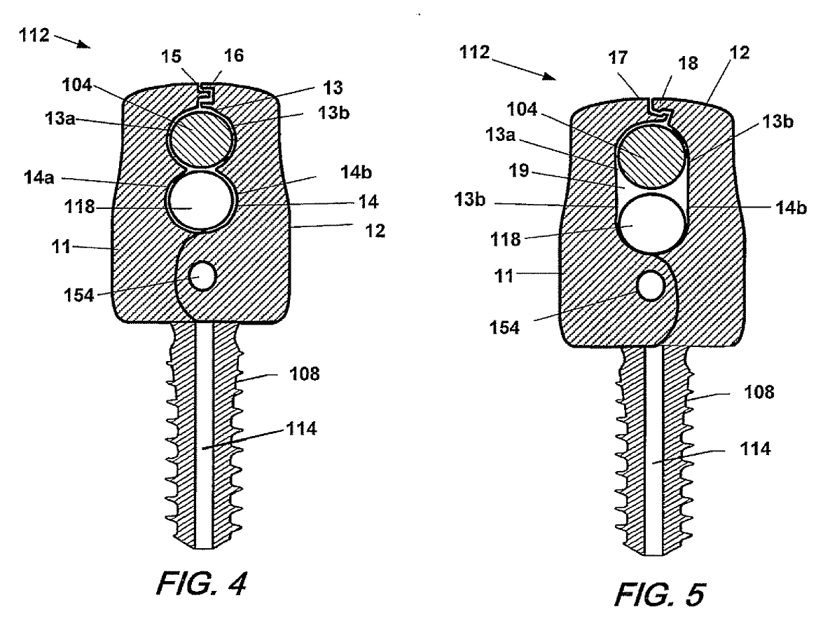

In some embodiments such as shown FIGS. 4 and 5, first member 11 may be hingedly joined to second member 12 using hinge 154. The hinged union of first member 11 and second member 12 may align first and second recessed portions 13a and 13b, and 14a and 14b, to form first cavity 13 and second cavity 14 (as depicted in FIG. 4) or to form a single cavity 19 (as depicted in FIG. 5) such that first cavity 13 is communicably joined to second cavity 14 and elongated member 104 contacts bone fastener 108.

In the embodiment depicted in FIG. 4, first member 11 having first recessed portion 13a and second recessed portion 14a may be hingedly joined using hinge 154 to second member 12 having first recessed portion 13b and second recessed portion 14b to form first cavity 13 and second cavity 14. First cavity 13 may be partially open to second cavity 14, such that contact between elongated member 104 and bone fastener 108 is possible but controlled in part by the orientation of cavity 13 with respect to cavity 14

Hinge 154 may be achieved using a hinge bracket and a hinge pin. Hinge 154 may be manufactured separately and joined to first member 11 and/or second member 12, or a hinge bracket and hinge pin may be integral to first member 11 and second member 12 to provide the same functionality. A hinge pin may rotate about a longitudinal axis of a hinge bracket such that the hinge pin may be capable of rotating a select angle about the hinge bracket, thereby rotatably connecting first member 11 and second member 12 about hinge 154 to form collar 112.

Collar 112 may further comprise a closure member. Closure members may be useful for capturing, locking, or otherwise maintaining the connection between first member 11 and second member 12 such that elongated member 104 and bone fastener 108 may be maintained in a selected configuration. In some embodiments, the closure member for connecting first member 11 to second member 12 may be internal, such as a rivet 15 for insertion into a cavity 16.

The embodiment depicted in FIG. 4 may provide a secure construct to maintain an elongated member 104 in a partially open configuration with respect to a bone fastener 108 without requiring a threaded closure member vertically threaded into collar 112 to maintain elongated member 104 in contact with bone fastener 108. One advantage of avoiding the use of threaded closure members to secure elongated member 104 in contact with bone fastener 108 is that the upper portion of the collar 112 (i.e. the part of collar 112 that extends above elongated member 104) may have a curved surface or otherwise less angular profile than prior art collars that require a cap or plug threaded to the collar. A second effect is that the collar may be manufactured with a lower profile than prior art collars, which typically require a minimum number of threads to securely engage a cap or other threaded closure member. FIG. 4 further depicts a cannulated configuration 114 of bone fastener 108 to enable minimally invasive procedures.

In FIG. 5, first member 11 may be hingedly joined using hinge 154 to second member 12 to form cavity 19. In this embodiment, elongated member 104 may be in contact with bone fastener 108 because the configuration or dimensions of first and second recessed portions 13a, 13b, 14a, and 14b may be combined into a single cavity 19.

A hinge bracket and a hinge pin may be manufactured separately and joined to first member 11 and/or second member 12 to form hinge 154, or a hinge bracket and a hinge pin may be integral to first member 11 or second member 12 to provide the same functionality. A hinge pin may rotate about a longitudinal axis of a hinge bracket such that the hinge pin may be capable of rotating a select angle about the hinge bracket, thereby rotatably connecting first member 11 and second member 12 to form collar 112.

Collar 112 may further include a closure member. Closure members may be useful for capturing, locking, or otherwise maintaining the connection between first member 11 and second member 12 such that elongated member 104 and bone fastener 108 are maintained in a selected configuration. In some embodiments, the closure member for connecting first member 11 to second member 12 may be internal or external. An extension that may be compression fit into a cavity may securely connect first member 11 to second member 12. In FIG. 5, an undercut area 17 may be configured to receive a portion of a tabbed extension 18.

The embodiment shown in FIG. 5 may provide a secure construct to maintain elongated member 104 in an open configuration with respect to bone fastener 108 without requiring a closure member superiorly threaded into collar 112 to maintain elongated member 104 in contact with bone fastener 108. One advantage of avoiding the use of threaded members to secure elongated member 104 in contact with bone fastener 108 is that the upper portion 31 of the collar (i.e. the part of collar 112 that extends above elongated member 104) may have a curved surface or otherwise less angular profile than prior art devices that require a cap or plug threaded to the collar. A second advantage is that the collar 112 may be manufactured with a lower profile than prior art collars, which typically require a minimum number of threads to securely engage a cap or other threaded closure member.

Advantageously, the use of a hinged connection enables embodiments of the present disclosure to be utilized in situations and settings not possible using prior art collars. For example, embodiments of the present disclosure may be positioned after bone fastener 108 and elongated member 104 have been positioned, in part because hingedly connected embodiments may be implanted by positioning one member first and rotating the second member about the hinge connection to engage the first member to form a collar while capturing the elongated member and bone fastener.

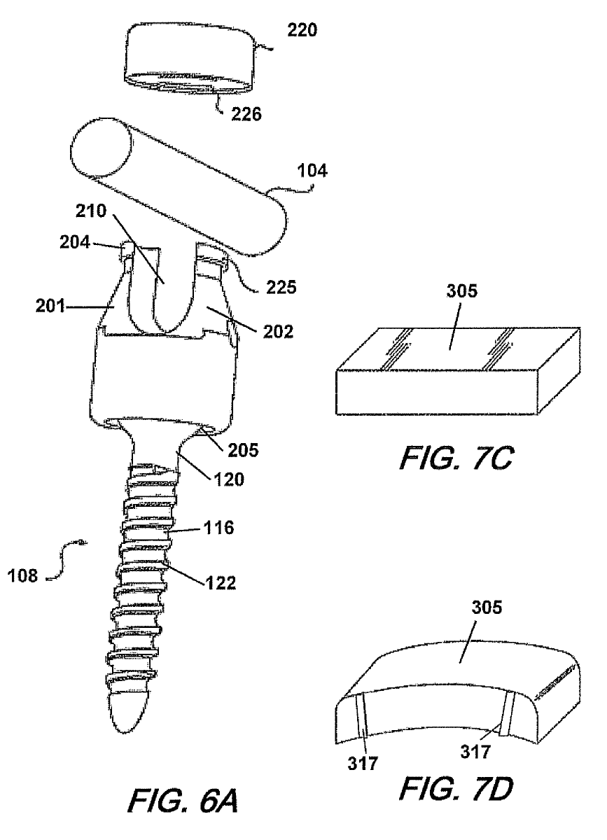

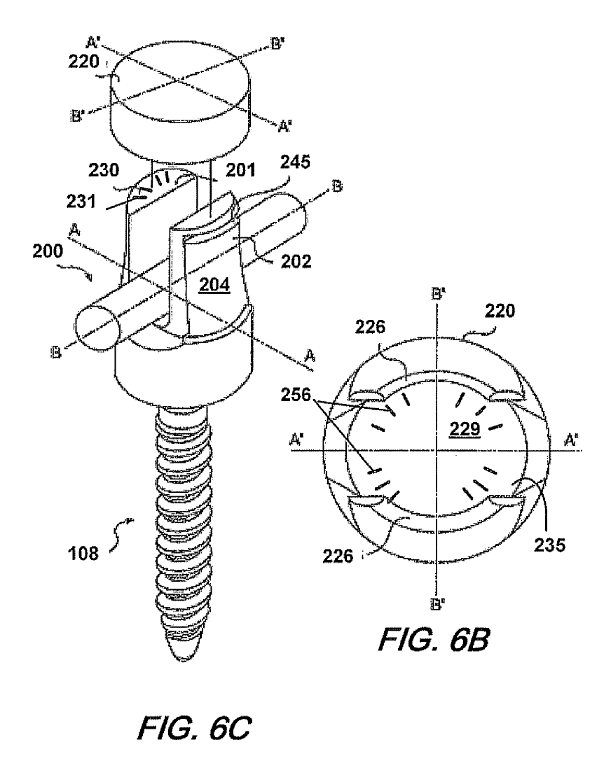

Now referring to FIGS. 6A-C, embodiments of the present disclosure may utilize a closure member featuring a cam-type mechanism for connecting to a collar to avoid the need for threaded closure members that are top-loaded in the collar to maintain the elongated member and bone fastener in a selected configuration. Advantageously, this type of closure member may be assembled using a lateral approach to the site and also may have a low-profile due to the avoidance of a threaded member for securing the elongated member to the bone fastener.

In an embodiment depicted in FIG. 6A, collar 200 having curved outer surface 204 may include a cavity or opening 205 having a selected depth and profile for receiving a portion of a bone fastener 108. Collar 200 may further include a channel 210 having a selected depth and profile for receiving a portion of an elongated member 104 in a selected configuration with the bone fastener 108. Channel 210 may further form two upwardly extending legs 201 and 202, and have a top surface for mating to a closure member 220. At least a portion of collar 200 may be non-circular.

FIG. 6B depicts a bottom view of an embodiment of a closure member 220 in which closure member 220 may function as a closure member for use in a cam-style mechanism for securing an elongated member (such as elongated member 104 in FIG. 6A) inside a collar (such as collar 200 in FIG. 6A). Closure member 220 may be configured having an inner surface 235 curved for selected engagement with a curved outer surface of a collar. Flanges 226 on the closure member 220 may be configured for slidable engagement of flanges 225 on a collar (as shown in FIG. 6A) or undercut 245 on a collar (as shown in FIG. 6C) to retain closure member 220 proximate to a collar. Closure member 220 may have an associated major axis (A′-A′) and minor axis (B′-B′). Advantageously, closure member 220 may enable surgeons to secure an elongated member to a collar without threading an insert or other threaded closure member onto threads in the collar. Closure member 220 may also have a curved upper surface for reduced profile.

Referring to FIGS. 6B and 6C, curved outer surface 204 of collar 200 and curved inner surface 235 of closure member 220 may have oval, elliptical, or some other non-circular profile having major and minor axes. In an embodiment, the action of rotating closure member 220 on collar 200 may function as a cam-style mechanism to retain closure member 220 on collar 200 and/or compress collar 200 to engage elongated member 104 in channel 210. In other words, when closure member 220 is oriented on collar 200 such that major axis A′-A′ (which has an associated major diameter) on closure member 220 is aligned with major axis A-A (with an associated major diameter) of curved surface 204 on collar 200, inner curved surface 235 on closure member 220 positioned about minor axis B′-B′ (with an associated minor diameter) on closure member 220 may not engage outer curved surface 204 of collar 200 positioned about minor axis B-B (with an associated minor diameter) on collar 200 and the construct is not assembled. As closure member 220 is rotated on collar 200 such that axis A′-A′ on closure member 220 approaches axis B-B on collar 200, inner curved surface 235 of closure member 220 may contact curved surface 204 of collar 200, and may compress legs 201 and 202 on collar 200 some amount based on the minor diameter of axis B′-B′. In embodiments having flanges 225 on closure member 220 and flanges 226 on collar 200, flanges 226 may engage flanges 225, thereby assembling the construct to securely retain elongated member 104 in relation to bone fastener 108. In addition, notches (not shown) on top surface 227 of flange 226 may engage notches (not shown) on the bottom surface 228 of undercut flange 225 to prevent rotation of closure member 220 about collar 200 once assembled. Also, indentations or protrusions 256 on bottom surface 229 of closure member 220 may engage notches 230 on top surface 231 of collar 200 to prevent undesirable rotation of closure member 220 about collar 200 once closure member 220 has been rotated into an assembled configuration on collar 200.

Advantageously, the use of a cam-style connection may enable embodiments of the present disclosure to be utilized in situations and settings not possible using prior art devices. For example, embodiments of the present disclosure may be positioned after bone fastener 108 and elongated member 104 have been positioned, in part because closure member 220 may be positioned and rotated on collar 200 from a lateral approach. Furthermore, closure member 220 may be connected to collar 200 without a significant vertical force applied to the spine.

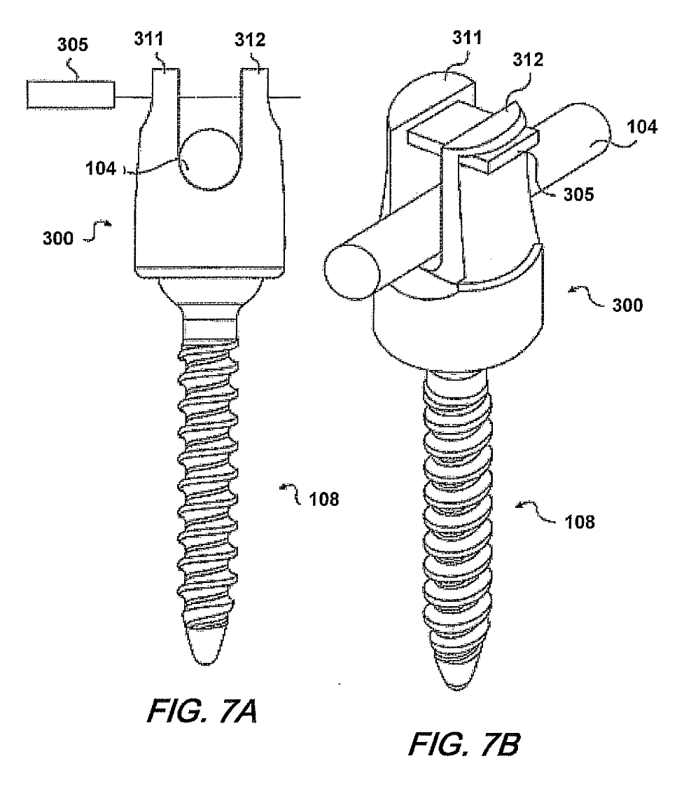

Now referring to FIG. 7A, embodiments of the present disclosure may utilize planar connector 305 inserted through rectilinear openings 306 in legs 311 and 312 to securely assemble elongated member 104 and collar 300 to bone fastener 108.

The components of collar 300 and planar connector 305 are preferably made from a suitable biocompatible material, such as titanium or stainless steel or other suitable metallic material, or ceramic, polymeric, or composite materials.

FIG. 7B depicts a perspective view of a planar connector 305 inserted through rectilinear openings 306 in arms 311 and 312 of collar 300 to provide selected contact with elongated member 104 such that elongated member 104 is in selected contact with the head of bone fastener 108.

One advantage to using a planar connector instead of a threaded member (e.g. screw or bolt) or cylindrical member (e.g. rivet) is that the contact changes from a point contact to a linear contact. In other words, prior art approaches generally have a cylindrical threaded member or rivet contacting a cylindrical elongated member, which forms a point contact, whereas embodiments of the present disclosure see advantages in having a planar connector contacting a cylindrical elongated member, which forms a linear contact. One advantage, for example, is that a curved planar connector is also possible for increased contact area, which is not possible using threaded member prior art approaches that must be straight. In some embodiments, planar connector 305 may have a generally straight profile as shown in FIG. 7C, or may have a selected curvature such as shown in FIG. 7D.

Connector 305 may be configured with extensions such as tabs 317 shown on FIG. 7D for engaging legs 311 and 312 on collar 300. Connector 305 or collar 300 or both may be configured for secure mechanical connection to maintain the connection between planar connector 305 and collar 300. For example, planar connector 305 and collar 300 may be configured for a compression fit or to be sweat-locked into position. Compression fit generally refers to embodiments in which the machine tolerances of the collar 300 and planar connector 305 are close or tight such that a large mechanical force is needed to bind them together. Compression fitting embodiments of the present disclosure is possible because the planar connector may be inserted lateral to the construct, so the large mechanical force would be applied tangent to the spine and not directed normal (or otherwise directed towards) the spine as applied using prior art approaches. Sweat-locking generally refers to the technique of cooling or heating pieces so that they may be assembled such that the subsequent heating or cooling due to implantation in the body results in a connection that requires large mechanical forces to separate the pieces.

Advantageously, the use of a planar connector connection enables embodiments of the present disclosure to be utilized in situations and settings not possible using prior art devices. For example, embodiments of the present disclosure may be positioned after bone fastener 108 and elongated member 104 have been positioned, in part because planar connector 305 may be positioned and inserted in collar 300 from a lateral approach.

Furthermore, embodiments as shown in FIGS. 7A-D may provide a secure construct to maintain an elongated member in a selected configuration with respect to a bone fastener without requiring a threaded member. One advantage of avoiding threaded members is that the upper portion 340 of the device (i.e. the part that extends above elongated member 104) may have a lower profile or define a curved surface.

The components of collar 300 and planar connector 305 may be manufactured from a suitable biocompatible material, such as titanium or stainless steel or other suitable metallic material, such as ceramic, polymeric, or composite materials.

In the foregoing embodiments, an elongated member 104 may be configured for connection to a collar 112, 200 or 300 attached to a bone fastener 108 that may be threaded or otherwise implanted in a vertebra, and/or to receive a connection for another component of a spinal implant system, such as, for example, a cross-link connection. In this regard, as is typical of spinal elongated members, elongated member 104 may be preferably solid with a uniform cylindrical shape that is compatible with a variety of collars 300 and/or connections. However, other configurations are possible, such as, for example, solid prismatic, elliptical, or helical shapes.

Collars according to embodiments of the disclosure may be used in minimally invasive surgery (MIS) procedures or in non-MIS procedures, as desired, and as persons of ordinary skill in the art who have the benefit of the description of the disclosure understand. MIS procedures seek to reduce cutting, bleeding, and tissue damage or disturbance associated with implanting a spinal implant in a patient's body. Exemplary procedures may use a percutaneous technique for implanting longitudinal rods and coupling elements. It is believed that the ability to implant the collar using MIS procedures provides a distinct advantage.

In the foregoing specification, the disclosure has been described with reference to specific embodiments. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

What is claimed is:

1. A collar for connecting an elongated member to a bone fastener comprising:
a first member having a recessed portion; and
a second member having a recessed portion configured for alignment with the first member, wherein the recessed portions form a cavity when aligned;
a hinge configured for rotatable connection between the first and second members; and
a closure member for fixed connection between the first and second members such that an elongated member is retained in a selected configuration with the bone fastener.

2. The collar of claim 1 wherein the first recessed portion is communicably coupled to the second recessed portion to define a channel and form two legs, wherein the two legs have interior surfaces configured for capturing a portion of the bone fastener and a portion of the elongated member.

3. The collar of claim 1, wherein an upper portion of the collar defines a substantially curved surface.

4. The collar of claim 1, wherein the closure member is an internal closure member.

5. The collar of claim 4, wherein the closure member comprises a rivet.

6. The collar of claim 1, wherein the closure member comprises an external closure member.

7. The collar of claim 1, wherein the closure member comprises a threaded member for engagement with a threaded cavity.

8. The system of claim 6, wherein the closure member comprises an undercut area for receiving a tabbed extension.

9. A collar for connecting an elongated member to a bone fastener comprising:
a curved outer surface having a major diameter and a minor diameter;
a cavity having selected depth and profile for receiving a portion of a bone fastener;
a channel having selected depth and profile for receiving a portion of an elongated member, wherein the sides of the channel form two upwardly extending legs; and
a closure member defining a curved inner surface with a major diameter greater than the major diameter of the curved outer surface of the collar and a minor diameter less than the major diameter of the curved outer surface of the collar,
wherein the action of rotating the closure member onto the collar compresses the legs inward to retain the elongated member in a selected configuration with the bone fastener.

10. The collar of claim 9, further comprising:
two or more flanges extending from the collar; and
two or more flanges extending from the closure member configured for rotatable engagement to the two or more collar flanges.

11. The collar of claim 9, further comprising:
two or more notches in the collar; and
two or more flanges extending from the closure member configured for rotatable engagement to the two or more collar notches.

12. The collar of claim 9, further comprising:
a plurality of radially extending features on the top surface of the collar; and
a plurality of radially extending features on the bottom surface of the closure member, wherein the radially extending features maintain the closure member in a selected orientation with the collar.

13. The collar of claim 9, wherein the closure member defines a curved upper surface.

14. The collar of claim 9, wherein an upper portion of the collar defines a curved surface.

15. A collar for connection between an elongated member and a bone fastener implanted in bony tissue, comprising:
a cavity of selected depth and profile for attachment to a portion of a bone fastener implanted in bony tissue;
a channel of selected depth and profile for receiving a portion of an elongated member, the channel forming two upwardly extending legs, wherein each leg comprises a rectilinear opening in alignment with the rectilinear opening on the opposite leg; and
a planar connector configured for insertion into the two rectilinear openings such that the elongated member is maintained in selected contact with the bone fastener.

16. The collar of claim 15, wherein the planar connector comprises a curved profile configured for selected contact with the elongated member.

17. The collar of claim 15, wherein the planar connector is compression fit into the two rectilinear openings.

18. The collar of claim 15, wherein the planar connector is sweat-locked into the two rectilinear openings.
